(19) United States
(12) Patent Application Publication
(10) Pub. No.: US 2023/0106599 A1
(43) Pub. Date: Apr. 6, 2023

(54) DIALYSIS MACHINE AND METHOD FOR RINSING SAME

(71) Applicant: PHYSIDIA, Saint-Barthélémy d'Anjou (FR)

(72) Inventors: Eric VINCENT, ANGERS (FR); Simon BRUNEAU, ANGERS (FR)

(21) Appl. No.: 17/800,529

(22) PCT Filed: Feb. 16, 2021
(86) PCT No.: PCT/FR2021/050269

§ 371 Date: Aug. 17, 2022

(30) Foreign Application Priority Data
Feb. 18, 2020 (FR) FR2001604

(51) Int. Cl. A61M 1/36

(52) U.S. Cl. CPC A61M 1/3649; A61M 1/365

(57) ABSTRACT

The invention relates to a dialysis machine comprising an arterial line (L1), a venous line (L2), a dialyser (100), a line for discharging (62) the dialysate, and a control unit (10) configured to perform a phase for rinsing the extracorporeal circuit by circulating physiological saline in the blood compartment of the dialyser and the arterial and venous lines, and a phase for eliminating air in the dialyser, which phase comprises stopping the circulation in the venous line (L2) and in the discharge line (62); and circulating dialysate from a dialysate supply source (40) through the blood compartment (12) and the arterial line (L1). The invention also relates to a method and a corresponding computer program product.

### FIELD OF THE INVENTION

The present invention relates generally to dialysis machines, in particular for hemodialysis, and to the use (operation and handling) of such machines.

### PRIOR ART

In general, hemodialysis consists of purifying the blood of substances that are present in excess, such as urea, vitamin B12, beta-immunoglobulin, or even mineral salts.

A dialysis machine usually comprises a dialysate circuit for circulation of dialysate and an extracorporeal circuit to which a patient is connected during a dialysis session. The dialysis machine also comprises a dialyzer which forms a connection interface between the dialysate circuit and the extracorporeal circuit. The dialyzer comprises a housing provided with a membrane which defines, within the housing, a blood compartment and a dialysate compartment.

The elimination of the substances present in excess in the patient's blood can be effected by osmosis, by circulating the blood on one side of the membrane and the dialysate on the other side of the membrane. Thus, the substances present in excess in the blood diffuse through the membrane into the dialysate. It is also possible to extract an aqueous fraction of the blood in an operation called ultrafiltration. In contrast to an ultrafiltration operation, it is also possible to introduce an appropriate quantity of dialysate into the blood in an operation called backfiltration.

Before a dialysis session is started, physiological saline is circulated in the extracorporeal circuit, which comprises the arterial and venous blood lines, and the blood compartment of the dialyzer, in order to rinse this extracorporeal circuit.

This circulation of physiological saline makes it possible to rinse and thus clean the extracorporeal circuit by removing the various particles or detritus that could lie in this extracorporeal circuit.

However, it is observed that air bubbles tend to accumulate in the upper part of the blood compartment of the dialyzer. These air bubbles increase the risk of clotting of the blood, on account of the contact between the blood, which circulates in this extracorporeal circuit during the dialysis session, and these air bubbles.

In addition, when these air bubbles are present at the level of the dialyzer membrane, the efficiency of the exchange surface between the blood compartment and the dialysate compartment is reduced.

For removing these bubbles, it has been found that users or operators may have a tendency to handle the dialyzer by turning it over or hitting it, in order to bring the bubbles back up into the bag of physiological saline.

However, such a practice is not desirable, since there is a risk of damaging the dialysis machine and possibly a risk of contamination.

The document WO2009055639 A2 describes a dialysis apparatus which comprises a dialysis instrument provided with a pump control device and with a first and a second valve control device, and with a disposable cassette compatible with the dialysis instrument. The disposable cassette comprises a pump part that can be controlled by the pump control device, and first and second valve chambers that can be controlled by the first and second valve control devices. The first and second valve chambers are in fluidic communication with each other.

The object of the present invention is to provide a novel dialysis machine and a novel corresponding rinsing method making it possible to overcome all or some of the problems set out above.

### SUMMARY OF THE INVENTION

To this end, the invention relates to a dialysis machine for treating a bodily fluid, such as blood or plasma, said machine having:
a dialyzer comprising an enclosure which includes:
a first passage zone for liquid, called a blood compartment, which has an inlet, called the bodily fluid inlet, and an outlet, called the bodily fluid outlet, and
a second passage zone for dialysate, called a dialysate compartment, which has a dialysate inlet and a dialysate outlet;
a membrane system between the first passage zone for liquid and the passage zone for dialysate;
an arterial line connected to the bodily fluid inlet of the blood compartment of the dialyzer; the arterial line having an end to which a first container, such as a bag, of physiological saline is connectable;
a venous line connected to the bodily fluid outlet of the blood compartment of the dialyzer; the venous line having an end to which a second container, such as a bag, is connectable;
a pump, called a blood pump, configured to allow a fluid to circulate back and forth in the arterial line;
a dialysate feed system comprising:
a dialysate feed line which is connected to the inlet of the dialysate passage zone, and
a dialysate delivery system which is connected to the feed line and to which a dialysate supply source is connectable;
a dialysate discharge system comprising a dialysate discharge line which is connected to the outlet of the dialysate compartment; the control unit being configured such that, in the connected state of the first container to the arterial line, of the second container to the venous line, and of the dialysate supply source to the dialysate delivery system, it implements a first rinsing phase, called an extracorporeal circuit rinsing phase, which comprises a step of control of the operation of the blood pump so as to cause the physiological saline, contained in the first container, to circulate into the second container;
characterized in that the control unit is configured to implement a phase of air elimination from the dialyzer, which comprises the following steps:
controlling the closure of circulation in the venous line;
controlling the closure of circulation in the discharge line of the dialysate discharge system;
controlling the operation of the dialysate delivery system in order to cause dialysate to circulate from the dialysate supply source through the blood compartment and the arterial line.

Such a design of the machine makes it possible to rinse the extracorporeal circuit of the dialysis machine while effectively eliminating the air bubbles liable to be blocked in the top of the dialyzer, and this without risk of damage and/or of contamination of the dialysis machine.

Moreover, the act of eliminating the bubbles by circulating dialysate from the dialysate supply source and in the direction of an ascent of the arterial line into the first container of physiological saline makes it possible to compensate at least in part for the quantity of physiological saline that has flowed from the first container to the second collection container during the rinsing phase.

It is thus possible to use a container of reduced volume, compared to that used in the prior art, to perform the rinsing step. The first container can then be a 1 liter bag instead of a 2 liter bag used in the prior art. Indeed, in the prior art, the bag had to be of sufficient capacity to retain a certain volume of physiological saline in anticipation of a return phase at the end of the dialysis session. With the invention, the dialysate that is collected in the physiological saline bag, by mixing with the physiological saline that remains after the rinsing phase, can be used in the return phase. It should be noted that the composition of the physiological saline is close to that of the dialysate.

The machine can also have one or more of the following features taken in any technically admissible combination.

According to one advantageous feature, the bodily fluid inlet of the dialyzer is situated above the bodily fluid outlet.

According to one advantageous feature, the venous line is provided with a clamp system, controllable by the control unit, to permit closing or opening of the circulation inside the venous line.

According to one advantageous feature, for execution of the air elimination phase, the control unit is configured to control the operation of the blood pump, in the opposite direction to the direction of operation used during the rinsing phase, in parallel with said step of controlling the operation of the dialysate delivery system.

According to one advantageous feature, with the dialysis machine comprising a pressure sensor arranged to measure the pressure in the dialysate circulation circuit formed between the pump and the dialysate delivery system, the control unit is configured to control the speed of operation of the pump as a function of the measured pressure so as to maintain said pressure within a predefined value range.

According to one advantageous feature, the control unit is configured such that, in the phase of rinsing and/or elimination of air, it varies the speed of operation of the pump in order to generate jolts.

According to one advantageous feature, said delivery system comprises at least one flexible bag, called a ventricle bag, intended to contain dialysate, and means for pressurizing the ventricle bag.

According to one advantageous feature, the control unit is configured to determine the volume of physiological saline displaced by the blood pump into the second container, and to stop operation of said blood pump when the volume of physiological saline displaced has reached a predefined value, for example between 600 and 1800 ml.

The invention also relates to a method of rinsing and eliminating air from a dialysis machine, said dialysis machine having:
a dialyzer comprising an enclosure which includes:
a first passage zone for fluid, called a blood compartment, which has an inlet, called the bodily fluid inlet, and an outlet, called the bodily fluid outlet, and
a second passage zone for dialysate, called a dialysate compartment, which has a dialysate inlet and a dialysate outlet;
a membrane system between the first passage zone for fluid and the passage zone for dialysate;
an arterial line connected to the bodily fluid inlet of the blood compartment of the dialyzer; the arterial line having an end to which a first container, such as a bag, of physiological saline is connectable;
a venous line connected to the bodily fluid outlet of the blood compartment of the dialyzer; the venous line having an end to which a second container, such as a bag, is connectable;
a pump, called a blood pump, configured to allow a fluid to circulate back and forth in the arterial line;
a dialysate feed system comprising:
a dialysate feed line which is connected to the inlet of the dialysate passage zone, and
a dialysate delivery system which is connected to the feed line and to which a dialysate supply source is connectable;
a dialysate discharge system comprising a dialysate discharge line which is connected to the outlet of the dialysate compartment; said method comprising a first rinsing phase, called an extracorporeal circuit rinsing phase, which comprises a step of control of the operation of the blood pump so as to cause the physiological saline, contained in the first container, to circulate into the second container,
characterized in that said method also comprises a phase of air elimination from the dialyzer, which comprises the following steps:
closure of circulation in the venous line;
closure of circulation in the discharge line of the dialysate discharge system;
operation of the dialysate delivery system in order to cause dialysate to circulate from the dialysate supply source through the blood compartment and the arterial line.

According to one advantageous feature, the phase of air elimination comprises operation of the blood pump, in the opposite direction to the direction of operation used during the rinsing phase, in parallel with said step of operating the dialysate delivery system.

The invention also relates to a non-transient computer program product comprising program code instructions for executing the steps of a method as described above, when said program is executed by a processor of a control unit of a dialysis machine as described above.

### BRIEF DESCRIPTION OF THE DRAWINGS

Other features and advantages of the invention will become more clearly apparent from the following description, which is purely illustrative and non-limiting and should be read with reference to the appended drawings, in which:

FIG. 1 is a schematic view of the extracorporeal circuit of a dialysis machine according to one embodiment of the invention, the dialysis machine being configured for a phase of rinsing the extracorporeal circuit;

FIG. 2 is a schematic view of the extracorporeal circuit of a dialysis machine according to one embodiment of the invention, the dialysis machine being configured for an air elimination phase (by reverse rinsing vis-à-vis the inlet of the dialyzer connected to the arterial line);

FIG. 3 is a schematic view of the dialyzer of a dialysis machine according to one embodiment of the invention, during an air elimination phase;

FIG. 4 is a view of the extracorporeal circuit and the dialysate circuit of a dialysis machine according to one embodiment of the invention and according to an operating configuration corresponding to a rinsing phase;

FIG. 5 is a view of the extracorporeal circuit and the dialysate circuit of a dialysis machine according to one embodiment of the invention and according to an operating configuration corresponding to an air elimination phase;

FIG. 6 is a flowchart showing steps of a rinsing method according to one embodiment of the invention.

### DETAILED DESCRIPTION

The concept of the invention is described more completely below with reference to the appended drawings, in which embodiments of the concept of the invention are shown. In the drawings, the size and the relative sizes of the elements may be exaggerated for reasons of clarity. Similar numbers make reference to similar elements throughout the drawings. However, this concept of the invention may be implemented in numerous different forms and should not be interpreted as being limited to the embodiments explained here. Instead, these embodiments are proposed such that this description is complete, and they communicate the extent of the concept of the invention to those skilled in the art. The embodiments which follow are examined, for the sake of simplification, in connection with the terminology and the structure of a dialysis machine and of a corresponding rinsing and air elimination method.

A reference throughout the specification to “a/one embodiment” means that a functionality, a structure or a particular feature described in connection with a/one embodiment is included in at least one embodiment of the present invention. Thus, the occurrence of the expression “in a/one embodiment” at various points throughout the specification does not necessarily make reference to the same embodiment. In addition, the functionalities, the structures or the particular features may be combined in any suitable manner in one or more embodiments. In particular, the order of the method steps may be changed when this remains technically admissible.

General Aspects

Referring to the figures and as mentioned above, the invention relates to a dialysis machine and a rinsing method for effectively rinsing the extracorporeal circuit of the dialysis machine before a dialysis session, by limiting the presence of bubbles in the extracorporeal circuit and in particular in the blood compartment of the dialyzer.

The dialysis machine makes it possible to treat a bodily fluid, such as blood or plasma. Bodily fluid means a fluid of the type present in the human or animal body, such as blood or plasma. In the remainder of the description, the bodily fluid considered is blood, but of course it could be another bodily fluid such as plasma.

According to one embodiment, the dialysis machine can comprise the same elements as those described in the international application numbered WO2013050689 A1 or WO2019150058 A2. However, the dialysis machine according to the invention is distinguished from those described in said international applications WO2013050689 A1 and WO2019150058 A2 in terms of a particular rinsing method implemented using computer and/or electronic instructions executed by a control unit, which is presented below.

Dialyzer

Said machine comprises a dialyzer 100. The dialyzer is also usually called a dialyzer filter. The dialyzer 100 is in the form of an enclosure which includes a passage zone 12 for bodily fluid, called a blood compartment, which has an inlet 201 for bodily fluid and an outlet 202 for bodily fluid. The enclosure also includes a dialysate passage zone 14, which has a dialysate inlet 401 and a dialysate outlet 402.

It should be noted that, in the rinsing method presented below, the fluid which circulates through the blood compartment 12 is not a bodily fluid but physiological saline or dialysate. However, the usual names of “blood compartment” and “bodily fluid” inlet/outlet or “blood pump”, which refer to use during a dialysis session, are retained.

The dialysate is a liquid well known to those skilled in the art, its composition being close to physiological saline. The dialysate comprises water of sufficient mineral and bacteriological quality. This water is added to a concentrate, in the form of an inorganic salt powder or in the form of a liquid concentrate, to make the required dialysate.

Physiological saline is usually composed of a mixture of water and sodium chloride. The dialysate is generally composed of physiological saline to which are added glucose and ions, such as potassium, magnesium, calcium and optionally lactate and/or bicarbonate according to the needs of the patient.

According to a preferred aspect, the dialysate used is sterile pyrogen-free dialysate. The dialysate has been sterilized in an autoclave, for example. This dialysate can be made from purified water and salt.

The circulation of dialysate passes through the dialysate compartment 14, generally counter to the flow of blood in the other blood compartment 12. The dialysate generally circulates in an open circuit. It is also possible to provide a partial regeneration loop of clean dialysate from the dialysate charged with toxins.

Membrane

The enclosure also includes a membrane system 3 to allow an exchange of elements (substances, molecules) between the bodily fluid, present in the passage zone 12 for bodily fluid, and the dialysate, present in the dialysate passage zone 14.

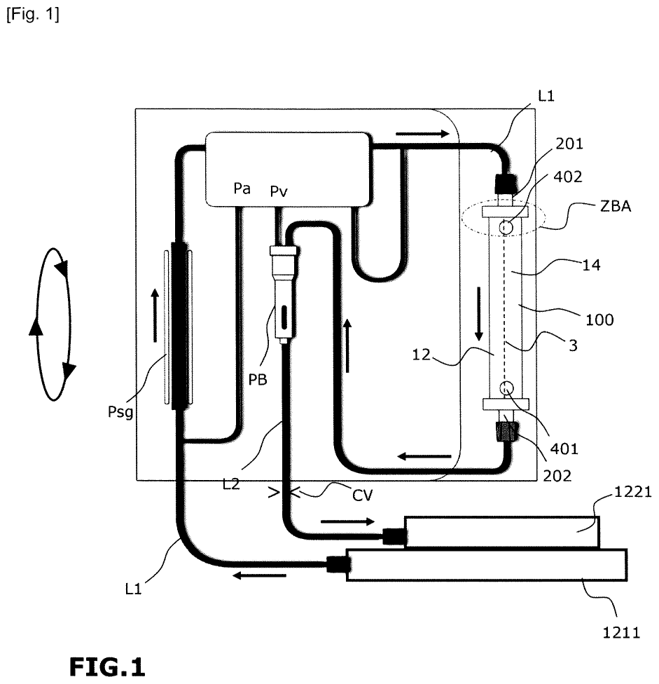

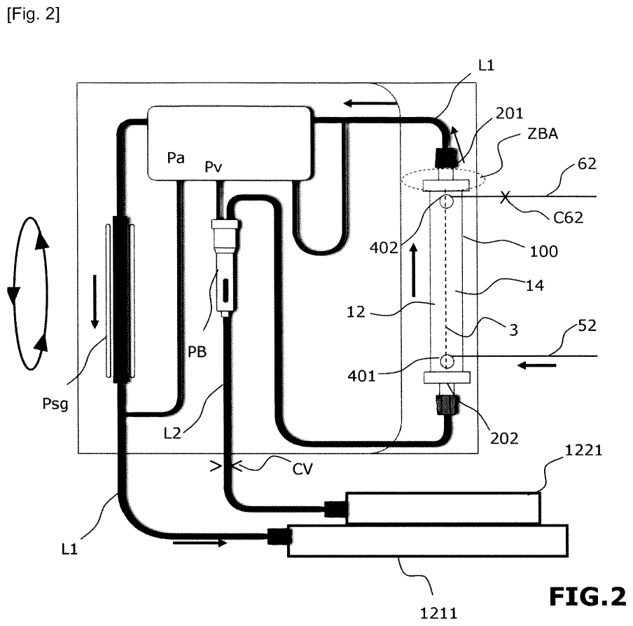

As is illustrated in FIGS. 1 and 2, from a functional point of view the dialyzer 100 can be schematized in the form of an enclosure housing a dialysis membrane 3 which separates the enclosure into a blood compartment, corresponding to the passage zone 12 for bodily fluid, and a dialysate compartment, corresponding to the dialysate passage zone 14.

The system with a semi-permeable dialysis membrane 3 is designed to allow a fraction of the blood volume to pass through when the difference between the local pressure in the blood compartment 12 and the local pressure in the dialysate compartment 14 is greater than a given value. This pressure difference is called the transmembrane pressure. In particular, the membrane system is designed such that only the smallest molecules are able to pass through the holes or pores of the membrane system, namely water, mineral salts and molecules of small to medium molecular weight.

More specifically, the membrane system 3 can comprise a membrane designed such that, when the difference between the pressure exerted on at least a portion of the membrane on the side of the blood compartment 12 and the pressure exerted on said portion of the membrane on the side of the dialysate compartment 14 is greater than a given threshold value, said portion of the membrane allows an aqueous fraction of the blood to pass into the dialysate compartment 14, according to a convective phenomenon called ultrafiltration. Conversely, said membrane 3 is designed such that, when the difference between the pressure exerted on said portion of the membrane 3 on the side of the dialysate compartment 14 and the pressure exerted on said portion of the membrane on the side of the blood compartment 12 is greater than a given threshold value, said portion of the membrane allows the dialysate to pass into the blood compartment 12, according to a phenomenon called backfiltration.

The dialysis membrane system can be provided in the form of capillary fibers, the blood circulating inside the fibers, the dialysate outside.

The blood inlet 201 is connected to an arterial line L1, one end of which, in order to carry out a dialysis session, is connected to a patient fistula, in order to be able to extract the blood from the body of the patient to be treated. Furthermore, the blood outlet 202 is connected to a venous line L2, one end of which is connected to a vein of the patient in order to reintroduce blood into the patient's body after treatment. The term “line” is understood to mean tubing which optionally comprises several branches or portions and through which a liquid can circulate. According to a particular aspect, these lines can be connected to each other such that one cannot be removed without the other.

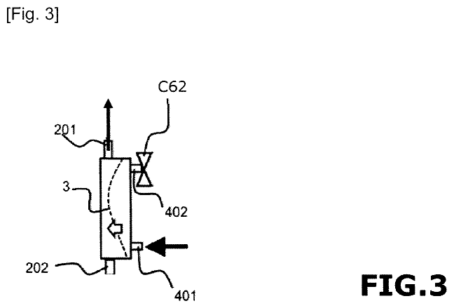

In the example illustrated in FIGS. 1 to 4, the arterial line L1 is also provided with a blood pump Psg and with a pressure sensor Pa, also called an arterial pressure sensor. The venous line L2 comprises a pressure sensor Pv, also called a venous pressure sensor, a bubble trap PB, and an air detector DA. Advantageously, the pressure sensor Pv is connected to the bubble trap PB. Preferably, said venous line L2 also comprises an anticoagulant injection system, such as a heparin pump PH.

The arterial line L1, the venous line L2 and the blood compartment 12 form the blood circuit of the machine.

Said dialysate compartment 14 has a dialysate inlet 401 and a dialysate outlet 402.

Dialysate Circuit

The machine also comprises a dialysate feed system 5 which comprises a dialysate feed line 52 which is connected to the inlet 401 of the dialysate compartment 14, and a dialysate discharge system 6 which comprises a discharge line 62 connected to the outlet 402 of the dialysate compartment 14.

Said dialysate feed system 5, the dialysate compartment 14, and said dialysate discharge system 6 form the dialysate circuit.

The dialysate feed line 52 is connected to the inlet 401 of the dialysate passage zone 14 by a quick connector, for example a Hansen type connector. Hansen type connectors correspond to the standard NF EN ISO 8637.

The feed system 5 also comprises a dialysate delivery system 51 connected to the feed line 52. Said dialysate feed system 5 allows dialysate to be delivered to the dialysate compartment 14 via the feed line 52.

Said delivery system 51 comprises at least one flexible bag 50, called a ventricle bag, intended to contain dialysate. Said system 51 can comprise several bags, as in the example illustrated in the figures.

Preferably, said dialysate feed system 5 comprises an additional ventricle bag 50′, which is mounted to bypass the portion of the supply line 52 to which said ventricle bag 50 is connected.

In the remainder of the description, reference is made to a system 51 comprising several bags 50, 50′, but it is understood that this description also applies to the case of a delivery system 51 comprising a single bag. Each ventricle bag of the system 51 is intended to contain dialysate and is connected to said feed line 52.

Each ventricle bag 50, 50′ is housed in a substantially sealed enclosure 500, 500′ which can be placed under pressure, and possibly under vacuum. Each ventricle bag 50, 50′ can thus be placed under pressure by pressurizing means 70 and, optionally, under vacuum by a vacuum generator 80.

Said pressurizing means 70 comprise a device for injection of pressurized gas, such as an air compressor or a cylinder of compressed gas, capable of injecting pressurized gas into the sealed enclosure 500, 500′ in which the ventricle bag 50, 50′ is housed. Said enclosure has an outlet, the opening of which is controllable by a solenoid valve in order to reduce the pressure in the enclosure.

The application of a pressure to the ventricle bag 50 or 50′ and the regulation of this applied pressure, with the aid of the pressure difference measured across the pressure drop tube 520, make it possible to maintain, for a given time, a fixed flow rate of dialysate in the dialysate compartment in a reliable and precise manner, and thus to control backfiltration operations. The application and the regulation of the pressure applied to the ventricle bag 50 or 50′ also make it possible, by adjusting the pressure applied to the discharge bag 60 or 60′, to maintain, for a given time, a given average pressure in the dialysate compartment, and thus to control ultrafiltration operations.

The two ventricle bags 50, 50′ are able to be placed under pressure/vacuum independently of each other.

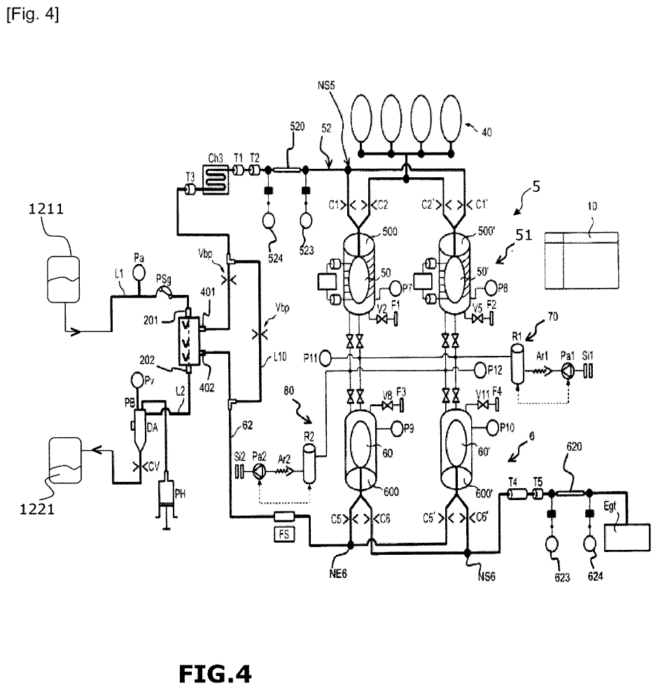

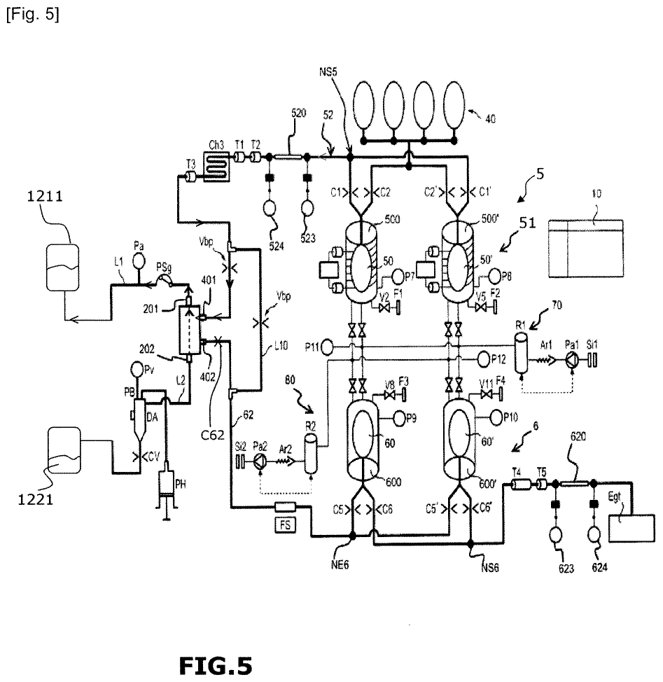

As is illustrated in FIG. 4 or 5, each ventricle bag 50, 50′ is also provided with a downstream closing/opening member C1, C1′ of the supply line which, in the open state, permits flow of dialysate from the corresponding bag 50, 50′ to the inlet 401 of the dialysate compartment, under the effect of a pressure applied to said ventricle bag by the corresponding means 70, and, in the closed state, prevents said flow of dialysate.

Each downstream closing/opening member C1, C1′ of the supply line is situated between the corresponding bag 50, 50′ and the outlet node NS5 for connecting the two bags 50, 50′. Each ventricle bag 50, 50′ is also provided with an upstream closing/opening member C2, C2′ which permits or prevents the supply to said ventricle bag 50, 50′ from a supply source 40. According to another embodiment, for example as described in the application WO2019150058 A2, it is possible to use a connection system of several supply sources, the additional source(s) being able to act as a reserve.

Each portion of the dialysate supply line 52 at which an upstream C2, C2′ or downstream C1, C1′ closing/opening member is located is flexible. Each of said closing/opening members C2, C2′, C1, C1′ is formed by a controllable clamp for, on the one hand, clamping the wall of said flexible portion of the supply line 52 from the outside so as to close this portion, and, on the other hand, for leaving open said flexible portion of said supply line 52.

The presence of the two ventricle bags and of the alternating clamp system formed by the members C1 to C2′ makes it possible to successively and alternately load the ventricle bags from the supply source 40. This makes it possible to continuously supply the dialysate feed line from one of the ventricle bags.

Placing the enclosure of a ventricle bag 50 or 50′ under vacuum with the aid of a vacuum generator 80 makes it possible, in the open state of the upstream opening/closing member C2 or C2′, and in the closed state of the corresponding downstream opening/closing member C1 or C1′, to aspirate the dialysate contained in the supply source 40 or 40′ in order to fill said ventricle bag 50 or 50′.

To control the flow of dialysate in the feed line 52, the control unit 10 controls the opening of the downstream member C1 or C1′ of one of the ventricle bags 50 or 50′ and the closure of the corresponding upstream member C2 or C2′. The use of two ventricle bags mounted in parallel allows one to be recharged while the other is in use.

The dialysate feed line 52 comprises a passage restriction 520 in order to create a pressure drop and to allow the pressure applied to the ventricle bag 50 or 50′ to be regulated as a function of the pressure difference measured across said restriction. In particular, said line 52 is provided with means of measuring the pressure difference between the inlet and the outlet of said passage restriction 520. This restriction 520 can be formed at least in part by a calibrated tube of constant cross section which forms a narrowing over a predetermined length. Said passage restriction 520 is situated between the bags 50, 50′ and the inlet 401 of the dialysate compartment 14.

The connection NS5 of the delivery system 51 to said feed line 52 is situated upstream of the passage restriction 520.

Said pressurizing means advantageously maintain a pressure on the ventricle bag 50 or 50′ that is sufficient to cause the flow of the dialysate. The feed line is calibrated such that, for a determined pressure applied to the ventricle bag 50 or 50′, the flow of dialysate in said line has a substantially constant value.

Such a system formed by a pressure drop tube and means for measuring the pressure difference across the restriction makes it possible in particular to determine the flow rate of dialysate which circulates in said feed line and which enters the dialysate compartment. By virtue of the dialysate flow rate thus measured, the control unit 10 can regulate the pressure applied to the ventricle bag 50 or 50′ in order to obtain the desired flow rate of dialysate entering the dialysate compartment and to precisely and reliably maintain this flow rate at a given value for a given time.

Said means for measuring the pressure difference across the restriction 520 of the feed line 52 can comprise two pressure tap orifices formed in the peripheral wall of said line 52, one upstream and the other downstream of said restriction 520. Each pressure tap orifice is associated with a pressure sensor 523, 524. Each pressure sensor 523, 524 is arranged relative to the corresponding pressure tap orifice in such a way as to measure the pressure in the feed line 52 at the level of said pressure tap orifice while being distanced from said orifice in order not to be in contact with the dialysate circulating in the line 52, 62.

The pressure sensors 523, 524 can be configured as described in the international application WO2013050689.

Provision can be made that the pressure measurement in the arterial line L1 and the venous line L2 is carried out in the same way as for the pressure measurement across the restriction 520. It is thus possible to provide that the pressure sensor Pa and the pressure sensor Pv are fixed in a part of the frame at a distance from a pressure orifice formed in the corresponding line L1, L2. According to a particular aspect, the pressure sensors Pa and Pv measure the pressure in the corresponding line L1, L2 without risk of contamination.

Dialysate Discharge System 6

The dialysate discharge system 6 comprises a dialysate discharge line 62 which is connected to the outlet 402 of the dialysate compartment 14, preferably by a quick connector, for example a Hansen type connector.

Said machine comprises a system, preferably of the clamp type, for opening/closing the dialysate discharge line 62. The opening/closing system, in the open state, permits the circulation in said discharge line 62 of the dialysate present at the outlet 402 of the dialysate compartment 14 for filling one of the discharge bags 60 or 60′, and, in the closed state, prevents the circulation of the dialysate in said discharge line 62 such that, for a sufficient pressure in the dialysate compartment, said dialysate passes through the membrane 3 into the blood compartment.

In the example illustrated in FIGS. 4 and 5, said dialysate discharge system 6 comprises at least one discharge bag 60, preferably flexible, having an inlet and an outlet that are connected to the dialysate discharge line 62. Said opening/closing system comprises an upstream opening/closing member C5 situated between the outlet 402 of the dialysate compartment 14 and said discharge bag 60. As is set out in detail below and shown in FIG. 5, there is also provided a closing/opening member C62 on the line 62.

Advantageously, said dialysate discharge system 6 comprises at least one other discharge bag 60′ mounted as a bypass of said discharge bag 60. Thus, the discharge bag 60 and the discharge bag 60′ are situated on two branches of the discharge line 62. In other words, starting from the outlet of the dialysate compartment 14, the discharge line 62 separates, upstream of the discharge bags 60, 60′, into two parallel branches, one provided for the discharge bag 60, the other for the discharge bag 60′. These two branches join each other downstream of the bags 60, 60′ and upstream of the cross-sectional restriction 620, as is set out in detail below.

Said opening/closing system comprises another upstream opening/closing member C5′ situated between said other discharge bag 60′ and the entry node NE6 for connecting the discharge bags 60, 60′.

Similarly to the upstream members C5, C5′, each discharge bag 60, 60′ is provided with a downstream closing/opening member C6, C6′ situated between the corresponding discharge bag 60, 60′ and the outlet node NS6 for connecting the discharge bags 60, 60′.

Each portion of the dialysate discharge line 62 at which an upstream C5, C5′ or downstream C6, C6′ closing/opening member is situated is flexible. Each of said upstream C5, C5′ or downstream C6, C6′ members for closing/opening the dialysate discharge line 62 is formed by a controllable clamp for, on the one hand, clamping the wall of said flexible portion of the discharge line 62 from the outside, so as to close this portion, and, on the other hand, for leaving open said flexible portion of said discharge line 62.

Such a set of upstream C5, C5′ and downstream C6, C6′ closing/opening members, associated with the discharge bags 60, 60′, makes it possible, in the open state of the upstream member C5, respectively C5′, and in the closed state of the downstream member C6, respectively C6′, to fill the discharge bag 60, respectively 60′, and, in the closed state of the upstream member C5, respectively C5′, and in the open state of the downstream member C6, respectively C6′, to empty said discharge bag 60, respectively 60′.

In the case where the discharge system 6 is formed by one or more discharge bags 60, 60′, the discharge line 62 is considered open when the dialysate is able to flow through said line in order to fill said bag or one of said bags.

The means for discharging the dialysate and the liquid present in the discharge bag 60 or 60′ can be formed by gravity if the configuration of the discharge bag lends itself to this and/or by pressurizing means 70, preferably common to those which are used to pressurize the ventricle bags 50, 50′. In the latter case, said or each bag 60, 60′ is housed in a substantially sealed enclosure 600, 600′ capable of being pressurized by said pressurizing means 70. Said enclosure has an outlet 601, the opening of which is controllable by a solenoid valve in order to reduce the pressure in the enclosure.

Provision can also be made that each enclosure 600, 600′ which houses a discharge bag 60, 60′ is connected to a vacuum generator 80. The vacuum generator 80 and/or the pressurizing means 70 make it possible to apply a pressure or a vacuum to the discharge bag 60 or 60′, so as to adjust the average pressure in the dialysate compartment 14, for example during an ultrafiltration phase. In this case, the downstream member C6 or C6′ of the discharge bag 60 or 60′ which is used to adjust the average pressure in the dialysate compartment 14 is closed while the corresponding upstream member C5 or C5′ is open.

Said vacuum generator 80 may be common for placing the enclosures of the dialysate feed system 5 under vacuum and for placing the enclosures of the discharge system 6 under vacuum.

Said machine comprises means for determining the quantity of dialysate and liquid recovered in the discharge bag 60 or 60′ which empties.

Said means for determining the quantity of dialysate and liquid recovered in the discharge bag(s) 60, 60′ comprise a passage restriction 620, formed in the dialysate discharge line 62 and situated downstream of said discharge bags 60, 60′, and means for measuring the pressure difference across said passage restriction 620.

Said means for measuring the pressure difference across the restriction 620 of the discharge line 62 may be similar to those associated with the restriction 520 of the feed line 52.

Thus, said means for measuring the pressure difference across the restriction 620 of the discharge line 62 can comprise two pressure tap orifices formed in the peripheral wall of said line 62, one upstream and the other downstream of said restriction 620. According to a particular aspect, each pressure tap orifice is associated with a pressure sensor arranged so as to measure the pressure in the line 62 at said orifice while being distanced from said orifice so as not to be in contact with the dialysate circulating in the line 62.

In a manner similar to the means for measuring the pressure difference across the restriction 520, a filter permeable to air and impermeable to infectious agents and to liquids can be interposed between each pressure sensor 623, 624 and the corresponding pressure tap orifice. In particular, each pressure sensor 623, 624 is mounted in a cavity of the frame of the machine with the aid of a hollow support piece which is intended to be coupled to the duct 621, 622 connected to the corresponding pressure tap orifice.

The pressure sensors 623, 624 associated with said restriction 620 make it possible to determine the pressure difference across the restriction 620 and thus the flow rate of dialysate and of liquid discharged from the discharge bag 60 or 60′, which makes it possible to determine, by measuring the corresponding flow time, the quantity of dialysate and of liquid recovered in said discharge bag 60 or 60′.

Control Unit

As has been mentioned above, said machine also comprises a control unit 10, such as a programmable logic controller. The control unit can be configured to execute dialysis steps, as are described in the international applications WO2013050689 A1 or WO2019150058 A2.

In addition to the initiation and return phases, a dialysis session also comprises a dialysis phase, which can comprise a plurality of ultrafiltration steps in alternation with backfiltration steps. An example of the operation of the machine during a dialysis session with a plurality of ultrafiltration steps in alternation with backfiltration steps is described in the international application WO2013050689 A1 or WO2019150058 A2. However, it should be noted that the dialysis phase generally comprises an exchange phase between the blood compartment and the dialysate compartment, for example by osmosis, and may not include a backfiltration phase and/or ultrafiltration phase.

For the dialysis phase, the control unit 10 controls in particular the means for pressurizing the bags 50, 50′ and the clamp systems C1, C2; C1′ C2′ and C5, C6; C5′, C6′ for closing/opening the supply system 5 and the discharge system 6. In a dialysis session, the initiation phase corresponds to the arrival of the blood in the blood compartment of the dialyzer. For this initiation phase, the control unit controls the blood pump Psg.

The dialysis session ends with a return phase during which the blood present in the blood circuit (venous line, arterial line, and the patient's blood compartment) is returned to the patient. For this return phase, the control unit also controls the blood pump Psg, a bag of physiological saline being connected to the line L1 in order to allow the blood to be returned without the risk of air being introduced.

As is set out in detail below, the control unit 10 is further configured to implement a method for rinsing and eliminating air from the extracorporeal circuit of the dialysis machine.

Said control unit 10 is in the form of an electronic and computer system which comprises, for example, a processor, such as a microprocessor, a working memory and a data memory. Said control unit may be in the form of a microcontroller.

In other words, the functions and steps described can be implemented in the form of a computer program or via hardware components (e.g. programmable gate arrays). In particular, the functions and steps performed by the control unit or by modules of the latter can be performed by sets of instructions or computer modules stored in a memory for their execution by a processor or controller or can be performed by dedicated electronic components or FPGA or ASIC type components. It is also possible to combine computer parts and electronic parts.

When it is specified that the unit or means or modules of said unit are configured to perform a given operation, this means that the unit comprises computer instructions and the corresponding means of execution to allow said operation to be performed and/or that the unit comprises corresponding electronic components.

Said control unit also has a data input interface. Provision can be made that said interface allows input of data relating to the patient and/or to the consumables of the machine.

Other Components or Means

In the example illustrated in the figures, the dialysis machine is also provided with conventional components to ensure reliable and efficient treatment of the bodily fluid to be treated, in particular a blood leak detector FS provided in the discharge line 62. Advantageously, the control unit 10 is configured to allow the flowmeters to be calibrated with respect to one another.

Each enclosure 500, 500′ is provided with means for heating or preheating the enclosure. Each enclosure 500, 500′, 600, 600′ is also provided with a vent valve V2, V5, V8, V11 associated with an air filter F1, F2, F3, F4, and a pressure sensor P7, P8, P9 and P10.

The pressurizing means 70 comprise the following elements: a compressed air tank R1, a pressure sensor P11 for the tank R1, a compressor Pa1, a non-return valve for the compressed air Ar1, and a compressed air filter and silencer Si1.

The vacuum generator 80 comprises an air vacuum tank R2, a pressure sensor P12 for the tank R2, a vacuum pump Pa2, a non-return valve for the air vacuum Ar2, and an air filter and silencer Si2 for the air vacuum.

The feed line 52 is also provided with means Ch3 for heating the dialysate associated with means for measuring temperature T1, T2, T3. The discharge line 62 is provided with means for measuring temperature T4, T5 downstream of the bags 60, 60′.

The venous line L2 also comprises a closing/opening member CV, such as a clamp.

The machine can comprise a bypass line L10, and also one or more associated closing/opening members Vbp. The bypass line L10 can be used to perform the initiation and calibration of the dialysate circuit, and to make it possible not to inject defective dialysate into the dialyzer, for example due to a temperature problem.

Advantageously, the discharge line of the dialyzer is connected to a recovery container or to a drainage system Egt.

Supply Source

The dialysate supply source used for a given dialysis session is preferably one or a set of feeder bags of greater volume, for example 5 liters, than that, for example 150 ml, of said ventricle bag 50, 50′.

Rinsing Method

An example of implementation of a rinsing method prior to a dialysis session is presented below.

The rinsing method is described with a dialysis machine as presented above, but it can be implemented with another dialysis machine.

A dialysate supply source 40 is connected to the dialysate feed system 5. The arterial line L1 is connected to the inlet 201 of the blood compartment 12 of the dialyzer 100, and the venous line L2 is connected to the outlet 202 of the dialysate compartment 14 of the dialyzer 100. The inlet 201 of the blood compartment 12 of the dialyzer 100 is situated above the outlet 202 of the dialysate compartment 14 of the dialyzer 100.

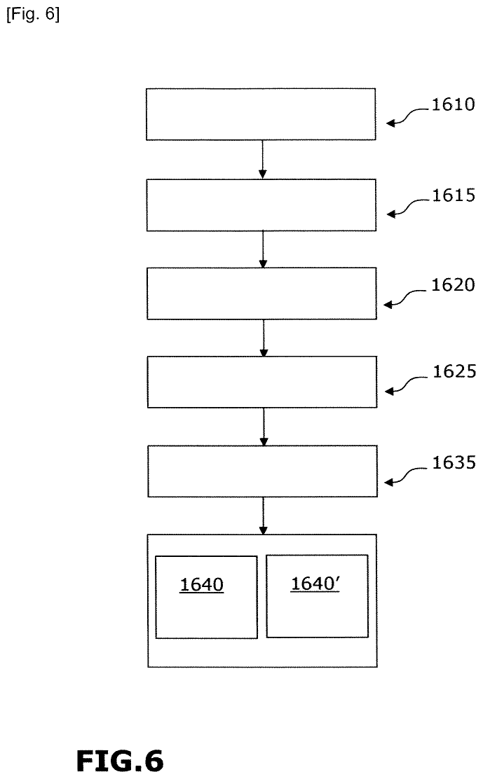

With reference to FIG. 1, to FIG. 4 and also to FIG. 6, which describes steps of a rinsing phase, the operator, who may be the patient, connects (step 1610) a bag 1211 of (sterile) physiological saline to an end of the arterial line L1 opposite the one connected to the dialyzer 100.

The end of the arterial line L1 to which the bag 1211 of physiological saline is connected may be a Y-shaped part comprising two connecting branches, of which a first branch is used for the connection of said bag 1211, and of which the second branch can be used subsequently for the connection of the patient. In particular, each of the first and second branches of the Y can be provided with a quick connector for respectively connecting thereto the bag 1211, then a connection device (for example an arteriovenous needle, a catheter, etc.) to the patient for arterial access, and a clamp system for controlling the closing/opening of the corresponding branch. The quick connector is, for example, a connector of the Luer Lock type, corresponding to the standard NF EN ISO 594.

In step 1615, the operator connects a collection bag 1221 to an end of the venous line L2 opposite the one connected to the dialyzer 100, which collection bag 1221 is intended to collect the physiological saline coming from the bag 1211 of physiological saline, as set out in detail below.

The end of the venous line L2 to which the bag 1221 for collecting physiological saline is connected can then be used for connection of the patient. The venous line can be provided with a quick connector, for example of the Luer Lock type, for connecting thereto the collection bag 1221, then a connection device (for example an arteriovenous needle, a catheter, etc.) to the patient for the venous return, and a clamp system for controlling the closing/opening of the corresponding branch.

First Rinsing Phase

A first rinsing phase is carried out in order to circulate physiological saline in the extracorporeal circuit. In summarized form, and as shown schematically with arrows in FIG. 4, during this first phase, physiological saline initially present in the bag 1211 of physiological saline passes into the arterial line L1, passes through the blood compartment 12 of the dialyzer, and passes into the venous line L2, entraining possible residues during its circulation, then is collected in the collection bag 1221. The clamp system CV, which makes it possible to close or open the circulation in the venous line L2, is opened in order to allow the physiological saline to be collected in the bag 1221.

In particular, and with reference to FIG. 1, FIG. 4 and FIG. 6, in step 1620 the control unit 10 controls the operation of the blood pump Psg in a given direction of rotation, so as to circulate the physiological saline from the bag 1211 of physiological saline into the collection bag 1221, inside which it is collected. The blood pump Psg can be a peristaltic pump, for example a pump as described in the application WO2013175115 A1.

This first rinsing phase corresponds to a rinsing of the extracorporeal circuit that permits removal of the physical and/or chemical residues present in the components, for example corresponding to material residues deriving from the manufacture of the components of the extracorporeal circuit.

Advantageously, for this rinsing phase, the control unit 10 controls the pump Psg so as to obtain a variation in the flow rate of physiological saline by alternating between 100 ml/min and 350 ml/min.

According to a particular aspect, the volume of physiological saline used during this first phase is between 600 and 1800 ml.

According to one embodiment, the control unit is configured to determine the volume of physiological saline that has passed, such that the control unit is able to determine when the volume of dialysate to be used for the first rinsing phase is reached and can carry out a second phase of air elimination, as presented below.

During this first rinsing phase, the circulation in the dialysate circuit can be closed, on the side of the dialysate feed system 5 and/or on the side of the dialysate discharge system 6. Alternatively, the circulation in the dialysate circuit can be opened. This is because the physiological saline flows from the bag 1211 into the bag 1221 with a low pressure, such that the physiological saline passes through the blood compartment 12 without passing through the membrane 3 in the dialysate compartment. Even if physiological saline were to pass into the dialysate compartment, this would not be a problem, since the physiological saline is, like the dialysate, a sterile liquid of a composition close to that of the dialysate.

After this first rinsing phase, the clamp system CV is closed at step 1625. The closing of the clamp system CV can be controlled by the control unit 10.

This circulation of the physiological saline makes it possible to rinse the extracorporeal circuit and thus to clean the latter by removing various particles or residues potentially present in the extracorporeal circuit, which are collected in the collection bag 1221.

However, air bubbles may have accumulated in the upper part, designated ZBA in FIGS. 1 and 2, of the blood compartment 12 of the dialyzer during this first rinsing phase.

Second Phase of Air Elimination

Thus, after the first rinsing phase presented above, a second phase of air elimination is carried out.

To carry out the second phase of air elimination, dialysate is circulated through the dialyzer 100 in the direction of an ascent of the arterial line L1, which is connected to the inlet 201 of the blood compartment 12 of the dialyzer 100, into the physiological saline bag 1211 which has been partially emptied during the first rinsing phase.

As has already been mentioned above, the circulation in the venous line L2 is closed.

The control unit 10 controls, in step 1635, the closure of the circulation in the dialysate discharge system 6, for example at the level of the discharge line 62. For this purpose, it is possible to provide that the control unit 10 controls the passage to the closed position of an opening/closing member C62 present on the discharge line 62, as is illustrated in FIGS. 2, 3 and 5. As a variant, the control unit 10 can control all or some of the other members C5, C5, C5′ and/or C6, C6′ so as to close the circulation in the discharge line 62. In particular, it is possible to simply close C5 and C5′.

Then, at step 1640, the control unit operates the dialysate delivery system 51 such that, with the circulation through the discharge line 62 being closed, the dialysate which enters the dialysate compartment 14 through the inlet 401 of the dialyzer 100 crosses the membrane 3 in order to pass into the blood compartment 12 and, with the venous line L2 being closed, rises through the inlet 201 in the arterial line L1 to the bag 1211 of physiological saline. The dialysate circulation is shown schematically with arrows in FIG. 5.

The dialysate thus travels through the arterial line L1 in the opposite direction to that used during the phase of rinsing with the physiological saline. The act of circulating the dialysate in the dialyzer 100 in the direction of an ascent of the arterial line L1 makes it possible to expel the air present in the upper part ZBA of the dialyzer, that is to say the air present on the side of the inlet 201 to which to the arterial line L1 is connected, as is illustrated in FIG. 1 and in FIG. 2.

Advantageously, the inlet 401 is situated below the outlet 402 of the discharge line 62 which is closed.

The blood pump Psg situated on the side of the extracorporeal circuit is also started up, at step 1640′, in parallel with the operation of the delivery system 51, in order to obtain, in the active part of the extracorporeal circuit (parts L1 and 12), a dialysate flow rate corresponding substantially to the dialysate flow rate supplied by the dialysate delivery system 51.

According to a particular aspect, when the dialysate delivery system 51 is operating, the blood pump Psg is controlled so as to provide a given flow rate (or setpoint flow rate) which is regulated as a function of the pressure measured in the active part of the extracorporeal circuit. Said pressure is preferably measured using the Pv sensor situated between the clamp system CV and the dialyzer 100.

The flow rate of the blood pump Psg is thus controlled such that the pressure in the active part of the extracorporeal circuit is within a predefined pressure value range, for example [0-100 mmHg] for a blood pump flow rate of 100 ml/min or [0-200 mmHg] for a blood pump flow rate of 350 ml/min.

When the measured pressure is greater than a threshold value, the blood pump Psg is then controlled so as to increase the flow rate in the arterial line L1 in order to bring the measured pressure back to the predefined value range, and, when the measured pressure is lower than a threshold value, the blood pump Psg is controlled so as to reduce the flow rate in order to bring the measured pressure back to a value within the predefined value range.

According to a particular aspect, the volume of dialysate used during this second phase of air elimination, by rinsing in the opposite direction, is chosen such that, at the end of the rinsing phase and of the air elimination phase, a given volume, for example 450 ml, remains in the physiological saline bag, in order to be able to ensure a return step at the end of the session.

In the rinsing and/or air elimination phase, provision can be made for the control unit 10 to abruptly vary the operating speed of the pump Psg in order to generate jolts during the circulation and thereby further improve the elimination of air in the extracorporeal circuit.

Once the air elimination phase is completed, the patient can then connect to the lines L1 and L2 in order to start a dialysis session.

In particular, the collection bag 1221 can be removed from the venous line L2 by opening the Luer Lock connector, and the patient connection device can be connected to this venous line L2 previously equipped with the collection bag 1221.

During the two steps of the rinsing process, the clamp system present on the branch of the Y-shaped system equipped with the physiological saline bag is open, and the one present on the other branch of the Y-shaped system is closed.

Before starting a dialysis session, the clamp system present on the branch of the Y-shaped system connected to the bag of physiological saline is closed.

At the end of the dialysis session, the clamp system of the branch of the Y-shaped system connected to the patient is closed, and the clamp system of the branch of the Y-shaped system connected to the bag of physiological saline is opened in order to ensure the return of the blood.

As has been mentioned above, when technically permissible, the order of certain steps can be reversed and the method of rinsing and air elimination can comprise, between two steps described, other additional steps.

The invention is not limited to the embodiments illustrated in the drawings. Furthermore, the term “comprising” does not exclude other elements or steps. In addition, features or steps that have been described with reference to one of the embodiments explained above may also be used in combination with other features or steps of other embodiments explained above.

1. A dialysis machine for treating a bodily fluid, including blood or plasma, said machine comprising:
a dialyzer having an enclosure which includes:
a first passage zone for liquid, called a blood compartment, which has an inlet, called the bodily fluid inlet, and an outlet, called the bodily fluid outlet, and
a second passage zone for dialysate, called a dialysate compartment, which has a dialysate inlet and a dialysate outlet;
a membrane system between the first passage zone for liquid and the passage zone for dialysate;
an arterial line connected to the bodily fluid inlet of the blood compartment of the dialyzer; the arterial line having an end to which a first container of physiological saline is connectable;
a venous line connected to the bodily fluid outlet of the blood compartment of the dialyzer; the venous line having an end to which a second container is connectable;
a pump, called a blood pump, configured to allow a fluid to circulate back and forth in the arterial line;
a dialysate feed system having:
a dialysate feed line which is connected to the inlet of the dialysate passage zone, and
a dialysate delivery system which is connected to the feed line and to which a dialysate supply source is connectable;
a dialysate discharge system having a dialysate discharge line which is connected to the outlet of the dialysate compartment;
a control unit being configured such that, in the connected state of the first container to the arterial line, of the second container to the venous line, and of the dialysate supply source to the dialysate delivery system, said control unit implements a first rinsing phase, called an extracorporeal circuit rinsing phase, which comprises a step of control of the operation of the blood pump so as to cause the physiological saline, contained in the first container, to circulate into the second container;
wherein the control unit is configured to implement a phase of air elimination from the dialyzer, which comprises the following steps:
controlling the closure of circulation in the venous line;
controlling the closure of circulation in the discharge line of the dialysate discharge system;
controlling the operation of the dialysate delivery system in order to cause dialysate to circulate from the dialysate supply source through the blood compartment and the arterial line.

2. The dialysis machine as claimed in claim 1, in which the bodily fluid inlet of the dialyzer is situated above the bodily fluid outlet.

3. The dialysis machine as claimed in claim 1, in which the venous line is provided with a clamp system, controllable by the control unit, to permit closing or opening of the circulation inside the venous line.

4. The dialysis machine as claimed in claim 1, in which, for execution of the air elimination phase, the control unit is configured to control the operation of the blood pump, in the opposite direction to the direction of operation used during the rinsing phase, in parallel with said step of controlling the operation of the dialysate delivery system.

5. The dialysis machine as claimed in claim 1, in which, with the dialysis machine comprising a pressure sensor arranged to measure the pressure in the dialysate circulation circuit formed between the pump and the dialysate delivery system, the control unit is configured to control the speed of operation of the pump as a function of the measured pressure so as to maintain said pressure within a predefined value range.

6. The dialysis machine as claimed in claim 1, in which the control unit is configured such that, in the phase of rinsing and/or elimination of air, said control unit varies the speed of operation of the pump in order to generate jolts.

7. The dialysis machine as claimed in claim 1, in which said delivery system comprises at least one flexible bag, called a ventricle bag, intended to contain dialysate, and means for pressurizing the ventricle bag.

8. The dialysis machine as claimed in claim 1, wherein the control unit is configured to determine the volume of physiological saline displaced by the blood pump into the second container, and to stop operation of said blood pump when the volume of physiological saline displaced has reached a predefined value.

9. A method of rinsing and eliminating air from a dialysis machine, said dialysis machine having:
a dialyzer having an enclosure which includes:
a first passage zone for liquid, called a blood compartment, which has an inlet, called the bodily fluid inlet, and an outlet, called the bodily fluid outlet, and
a second passage zone for dialysate, called a dialysate compartment, which has a dialysate inlet and a dialysate outlet;
a membrane system between the first passage zone for liquid and the passage zone for dialysate;
an arterial line connected to the bodily fluid inlet of the blood compartment of the dialyzer; the arterial line having an end to which a first container of physiological saline is connectable;
a venous line connected to the bodily fluid outlet of the blood compartment of the dialyzer; the venous line having an end to which a second container is connectable;
a pump, called a blood pump, configured to allow a fluid to circulate back and forth in the arterial line;
a dialysate feed system having:
a dialysate feed line which is connected to the inlet of the dialysate passage zone, and
a dialysate delivery system which is connected to the feed line and to which a dialysate supply source is connectable;
a dialysate discharge system having a dialysate discharge line which is connected to the outlet of the dialysate compartment,
said method comprising the steps of:
a first rinsing phase, called an extracorporeal circuit rinsing phase, which comprises a step of control of the operation of the blood pump so as to cause the physiological saline, contained in the first container, to circulate into the second container,
wherein said method also comprises a phase of air elimination from the dialyzer, which includes the following additional steps:
closure of circulation in the venous line;
closure of circulation in the discharge line of the dialysate discharge system;
operation of the dialysate delivery system in order to cause dialysate to circulate from the dialysate supply source through the blood compartment and the arterial line.

10. The method as claimed in claim 9, in which the phase of air elimination comprises operation of the blood pump, in the opposite direction to the direction of operation used during the rinsing phase, in parallel with said step of operating the dialysate delivery system.

11. A non-transient computer program product comprising program code instructions for executing the steps of a method as claimed in claim 9, when said program is executed by a processor of a control unit being configured such that, in the connected state of the first container to the arterial line, of the second container to the venous line, and of the dialysate supply source to the dialysate delivery system, said control unit implements a first rinsing phase, called an extracorporeal circuit rinsing phase, which comprises a step of control of the operation of the blood pump so as to cause the physiological saline, contained in the first container, to circulate into the second container;
wherein the control unit is configured to implement a phase of air elimination from the dialyzer, which comprises the following steps:
controlling the closure of circulation in the venous line;
controlling the closure of circulation in the discharge line of the dialysate discharge system;
controlling the operation of the dialysate delivery system in order to cause dialysate to circulate from the dialysate supply source through the blood compartment and the arterial line.

12. The dialysis machine as claimed in claim 1, wherein said first and second containers are bags.

13. The dialysis machine as claimed in claim 8, wherein said predefined value is between 600 and 1800 ml.

14. The method as claimed in claim 9, wherein said first and second containers are bags.
